(19) United States
(12) Patent Application Publication
(10) Pub. No.: US 2025/0319080 A1
(43) Pub. Date: Oct. 16, 2025

(54) BIODEGRADABLE COMPACTED FORMULATIONS AND METHODS OF USE AND MANUFACTURE THEREOF

(71) Applicant: PURDUE RESEARCH FOUNDATION, West Lafayette, IN (US)

(72) Inventors: Andrew David Otte, West Lafayette, IN (US); Kinam Park, West Lafayette, IN (US)

(21) Appl. No.: 19/250,827

(22) Filed: Jun. 26, 2025

Related U.S. Application Data
(63) Continuation-in-part of application No. 18/538,022, filed on Dec. 13, 2023.
(60) Provisional application No. 63/435,260, filed on Dec. 25, 2022.
(60) Provisional application No. 63/779,808, filed on Mar. 28, 2025.

(51) Int. Cl. A61K 31/485; A61K 9/16; A61K 9/20

(52) U.S. Cl. CPC A61K 31/485; A61K 9/1647; A61K 9/204; A61K 9/2095

(57) ABSTRACT

The present disclosure generally relates to formulations comprising a drug and biodegradable polymers that are compacted mechanically from a physical mixture to disrupt interconnected pores or open channels, resulting in substantially longer drug release times compared to non-compacted counterparts. The disclosure also includes methods of use and manufacture thereof.

### RELATED APPLICATIONS

This application claims the benefit of U.S. Provisional Application No. 63/779,808, filed Mar. 28, 2025, and is a Continuation in Part of U.S. application Ser. No. 18/538,022, filed Dec. 13, 2023, which claims the benefit of U.S. Provisional Application No. 63/435,260, filed Dec. 25, 2022, the entire disclosure of each of which is incorporated herein by reference.

### GOVERNMENT SUPPORT

This invention was made with government support under Grant No. UG3DA059270 and UH3DA048774 awarded by the National Institutes of Health/National Institute on Drug Abuse. The government has certain rights in the invention.

### FIELD OF INVENTION

The disclosure generally relates to biodegradable, highly compacted formulations and methods of use and manufacture thereof.

### BACKGROUND

Opioid use disorder (OUD) has become an epidemic in the United States, and accessible OUD treatments are urgently required. Untreated OUD, a chronic brain disease, has a serious cost to people, their families, and society. During 2022-2024, more than 300,000 people died due to drug overdose. Each year, opioid overdose, misuse, and dependence are estimated to account for more than $100 billion in healthcare costs, criminal justice costs, and lost productivity. Mortality related to OUD has been reduced by opioid agonist therapy, including buprenorphine and methadone. Methadone has higher side effects than buprenorphine. Buprenorphine is a partial agonist of the μ-opioid receptor, producing effects such as euphoria or respiratory depression at low to moderate doses, and has been used to treat OUD with counseling and behavioral therapies. Buprenorphine treatment is known to improve the overall physical, psychological, and social quality of life for individuals with OUD.

The buprenorphine products approved by the U.S. Food and Drug Administration (FDA) for the treatment of OUD include oral and injectable formulations. Sublingual tablets and films have a considerable diversion of the medications and risks of non-adherence. Extended-release depot injection of buprenorphine can prevent diversion, daily fluctuations in plasma concentrations, poor daily adherence, medication misuse, and accidental poisoning in children and possibly provide improvements in treatment retention.

### SUMMARY

The present disclosure recognizes that providing additional and longer-acting drug (e.g., buprenorphine)-based formulations to patients and physicians is a potential, straightforward method that can improve treatment retention. Long-acting formulations are typically prepared using biodegradable poly(lactide-co-glycolide), also referred to as poly(lactic-co-glycolic acid), which is abbreviated as PLGA. Understanding of long-acting PLGA formulations and the drug release mechanisms provides a potentially breakthrough technology that can be readily applied to long-acting formulations requiring high drug loading with controlled release kinetics. Compacted single-rod and compacted microgranule formulations, as described herein, require control of fewer processing variables that are readily scalable and potentially have a lower cost than microparticles prepared by traditional emulsion methods. Here, microgranules refer to granules obtained by grinding larger compacted objects, such as compacted rods, matrices, or plates, and they are distinguished from microparticles obtained through conventional emulsion-based methods. The compacted single-rod and microgranule platforms can overcome the typical problems associated with other formulations, such as emulsion-based PLGA microparticle formulations, which often exhibit low drug loading and high initial burst release if not formulated correctly. Compacted rods and microgranules offer an ideal vehicle for high drug loads, scalable manufacturing with low cost, and extended therapy durations. The increased microstructural density of the compacted rods and microgranules, with reduced interconnected pores, allows for slower water uptake kinetics, ultimately resulting in slower drug release.

To that end, and in certain aspects, the disclosure provides biodegradable, implantable and injectable formulations comprising a drug and one or more biodegradable polymers, wherein the formulation is essentially free of interconnected pores.

In other aspects, the disclosure provides methods of treating a subject with an OUD that involve implanting or injecting in a subject a biodegradable implantable or injectable formulation comprising a drug that treats opioid abuse and one or more biodegradable polymers, wherein the formulation is essentially free of interconnected pores.

In other aspects, methods of making a biodegradable formulation that is substantially free of pores and that involves providing a mixture of a drug and one or more biodegradable polymers, compacting the mixture with sufficient mechanical force to remove surface pores and a substantial amount of internal pores; and applying heat to sustainably remove remaining internal pores, thereby producing a biodegradable formulation that is substantially free of interconnected pores.

In certain embodiments of the formulations and methods herein, the biodegradable formulation comprises about 40 to 80% by weight of the drug. In certain embodiments of the formulations and methods herein, the biodegradable formulation comprises about 50 to 70% by weight of the drug.

In certain embodiments of the formulations and methods herein, the biodegradable formulation comprises about 20 to 60% by weight of biodegradable polymer. In certain embodiments of the formulations and methods herein, the biodegradable formulation comprises about 30 to 50% by weight of biodegradable polymer. In certain embodiments of the formulations and methods herein, the biodegradable polymer is poly(lactide-co-glycolide), poly(D,L-lactide), poly(ε-caprolactone), polyhydroxybutyrate, polyanhydrides, polyorthoesters, or combinations of any of these. In certain embodiments of the formulations and methods herein, the biodegradable formulation provides sustained release of the drug for about 90 days or longer. In certain embodiments of the formulations and methods herein, the biodegradable formulation has a surface that is free of interconnected pores.

In certain embodiments of the formulations and methods herein, the drug is buprenorphine. In certain embodiments of the formulations and methods herein, the buprenorphine is of the free base form, salt form, or mixtures thereof.

### BRIEF DESCRIPTION OF THE DRAWINGS

FIG. 1 illustrates a schematic diagram of drug/excipient rod formation using a plastometer (also referred to as an extrusion plastometer or a melt flow index tester).

FIG. 2 shows (from bottom to top) powder x-ray diffraction patterns of buprenorphine HCl from the Cambridge Structural Database (CSD), buprenorphine HCl tested as received, buprenorphine free base (FB) from the CSD, and buprenorphine FB prepared.

FIG. 3 shows the particle size distribution of cryo-milled PLGA and buprenorphine free base. IV indicates the inherent viscosity (dL/g) of PLGAs used.

FIG. 4 shows the in vitro release plots of the buprenorphine rods in Table 4.

FIG. 5 shows rodent pharmacokinetic profiles of buprenorphine rods in Table 4.

FIG. 6 shows pharmacokinetic profiles of buprenorphine rods in Table 6 tested in dogs.

FIG. 7 shows the in vitro release of buprenorphine rods in Table 8 in 0.5% SDS (Top) and 0.05% PBST (Bottom).

FIG. 8 shows pharmacokinetic profiles of buprenorphine rods in Table 9 in dogs.

### DETAILED DESCRIPTION

The present disclosure relates to implantable and injectable, controlled-release (or sustained-release) compacted formulations comprising a drug (e.g., buprenorphine or a salt thereof) and a biodegradable polymer such as PLGA.

It is highly beneficial to understand the drug release mechanisms to develop formulations with the intended drug release profiles. Trial-and-error approaches, typically used in prior formulation development, offer no scientific basis for understanding and improving drug release kinetics. In particular, the initial burst release is ubiquitous in current FDA-approved formulations, and its causes are not clearly understood despite various theories having been proposed; thus, it is difficult to prevent (Park, PLGA-based long-acting injectable (LAI) formulations, J. Control. Release, 382: 113758, 2025).

Recent studies indicate that the initial burst release is due to rapid absorption of water into PLGA formulations and subsequent rapid release of the loaded drug (Park et al., Injectable, long-acting PLGA formulations: Analyzing PLGA and understanding microparticle formation, J. Control. Release, 304: 125-134, 2019; Park et al., Potential roles of the glass transition temperature of PLGA microparticles in drug release kinetics, Mol. Pharm. 18: 18-32, 2021; Otte et al., The impact of post-processing temperature on PLGA microparticle properties, Pharm. Res. 40: 2677-2685, 2023). The initial burst release is followed by the rearrangement of PLGA polymers at 37° C., close to their glass transition temperature (T_{g}), which forms a PLGA membrane on the surface and controls drug release at the steady state. The initial fast absorption of water upon exposure of PLGA formulations to water is due to the presence of interconnected pores in the formulations, which are formed as a result of removing the solvent used in making the emulsion-based PLGA formulations.

The interconnected pores are also formed in formulations prepared by the melt extrusion process. As the hot, molten drug/PLGA mixture is extruded, it expands due to the recovery of polymer molecules from an aligned and deformed structure to relaxed random coil structures, a phenomenon known as die swell. Even after cooling back to the room temperature, the diameter remains larger than that of the die orifice. The melt extrusion process has been used for preparing PLGA-based solid implants, e.g., ZOLADEX DEPOT delivering 10.8 mg goserelin acetate for 3 months, OZURDEX delivering 0.7 mg dexamethasone for 3 months, PROPEL delivering 0.37 mg mometasone furoate for 1 month, SCENESSE delivering 16 mg afamelanotide for 2 months, and DURYSTA delivering 0.01 mg bimatoprost for 6 months. A hot melt extrusion process using a molten mass of PLGA was used to prepare about 47.5% w/w buprenorphine-loaded implants in the presence of a large amount of a lubricant, e.g., 1-15% w/w of glyceryl monostearate (Saxena, K. and Saxena, N. A biodegradable implant composition and process for long-term delivery of buprenorphine and use thereof, International Application Number PCT/IN2022/050137, International Publication Number WO 2022/175977 A1, 2022).

Sometimes, the poor water solubility of a drug results in moderate initial burst release. For most drugs, an important factor in preventing the initial burst release is to minimize the interconnected pores. Eliminating such open pores in a biodegradable matrix can provide certain advantages for the formulations.

Furthermore, the duration of buprenorphine release from emulsion-based PLGA 50:50 microparticles with only ≤5% of the drug loading is only 3 days (Schreiner et al., Design and in vivo evaluation of a microparticulate depot formulation of buprenorphine for veterinary use, Sci Rep. 10:17295, 2020). SUBLOCADE (buprenorphine extended release, Indivior), an in situ forming implant (ISFI) with 20% buprenorphine loading in PLGA 50:50, releases the drug for 1 month (Sublocade 2017, package insert). An in situ forming gel, composed of a PLGA-PEG-PLGA triblock copolymer, was used for delivery of buprenorphine for 1 month (PLGA 75:15) (Kamali et al., In-vitro, ex-vivo, and in-vivo evaluation of buprenorphine HCl release from an in situ forming gel of PLGA-PEG-PLGA using N-methyl-2-pyrrolidone as solvent, Materials Science and Engineering: C, 96:561-575, 2019). The compacted single-rod and microgranule formulations absorb water more slowly, resulting in slower drug release and slower degradation compared to conventional formulations. Thus, even PLGAs with a 50:50 L:G ratio can release buprenorphine for ≥3 months.

The injectable buprenorphine formulations are typically configured into one of three delivery systems: microparticle (MP), injectable ISFI (e.g., Sublocade), or solid PLGA implant. ISFIs have not been demonstrated to be clinically useful for 3-month buprenorphine delivery to date. Microparticle formulations can deliver buprenorphine for 3 months or longer by optimizing formulation variables and processing conditions for scale-up manufacturing. Microparticle formulations, however, can encounter issues during scale-up, such as the use of large quantities of organic solvents and controlling the multiple parameters that dictate drug loading and release. Solid PLGA implants have been typically used for the delivery of small amounts of drugs, e.g., ≤16 mg in FDA-approved products.

Understanding the drug release mechanisms of long-acting PLGA formulations provides a technology that can be applied to developing long-acting formulations requiring high drug loading and ≥3 months of drug release kinetics. The compacted single-rod and microgranule methods described herein require control of considerably fewer processing variables, are readily scalable, and potentially have a lower cost than microparticles prepared by traditional emulsion methods. The compacted rod platform can overcome typical problems associated with other formulations, such as PLGA microparticle formulations with low drug loading and high initial burst release if not formulated correctly. Compacted rods and microgranules offer an ideal platform for high drug loads, simple processing, scalable manufacturing, and extended therapy durations. The increased microstructural density of the compacted rods and microgranules allows slower water uptake kinetics, ultimately resulting in low initial burst release and slower drug release. Key design parameters for formulations with a delivery duration of ≥3 months are described below.

The compaction of drug-PLGA powder mixtures involves a compaction step that results in a reduction in volume by displacing the gaseous phase, followed by polymer entanglement and/or plastic deformation and consolidation (assembly into a single object) or fusion. The drug-PLGA mixture can include other pharmaceutical excipients, such as a binder. The primary purpose of compaction is to displace the gaseous phase and minimize interconnected pores or open channels, thereby controlling drug release and extending the duration of drug release for several months. Optimal compaction requires a careful selection of PLGAs with the correct particle size and density to achieve the desired dimensions of the punch and die. Other biodegradable polymers include, but are not limited to, poly(D,L-lactide), poly(ε-caprolactone), polyhydroxybutyrate, polyanhydrides, polyorthoesters, or combinations of any of these.

The commercial failure of the 6-month Probuphine formulation is largely due to the surgical insertion of 4 (and up to 5) implants, followed by their surgical removal after 6 months. Probuphine was produced by hot-melt extrusion of a buprenorphine HCl and non-biodegradable ethylene-vinyl acetate (EVA) copolymer blend, resulting in an implant with a diameter of 2.4 mm (Kleppner et al., In-vitro and in-vivo characterization of a buprenorphine delivery system, J. Pharm. Pharmacol., 58: 295-302, 2006). To fabricate implants, buprenorphine was dry-blended with EVA copolymer, followed by melt extrusion to form a fiber ˜2.4 mm in diameter, and then cut into implants 26 mm in length. The implants were washed in 95% ethanol at room temperature for 30 minutes to remove the surface drug and thus minimize the initial release of buprenorphine. The relative amount of buprenorphine lost during this washing step is uncertain. The washed implants were air-dried at room temperature for 30 min, then forced-air-dried at 40° C. for 1 hour, followed by vacuum drying at 30° C. for 24 hours to remove residual ethanol. In this non-biodegradable implant, water molecules permeate through the interconnected pores, resulting in the release of buprenorphine. Release from non-biodegradable implants is primarily determined by the surface area, the rate of drug dissolution, and the diffusion of the drug through the polymeric matrix.

Our long-acting buprenorphine formulations described here provide novel and valuable improvements over prior art formulations. These improvements include the following. The polymer used is biodegradable, so surgical removal is not necessary at the end of the product's use. In the event that surgical removal is required due to discontinuation of the desired therapy, the compacted rod product may be removed for patients who elect to discontinue treatment. The PLGA type is judiciously selected to control the duration and kinetics of drug release. The PLGA formulation is substantially devoid of interconnected pores, which can minimize the initial burst release and maintain the desired steady-state release kinetics for extended periods. The formulation is preferably provided in a single configuration, and the drug loading is sufficiently high to allow for administering only one implant. The formulation is preferably formulated in a manner that provides for its manufacture under current Good Manufacturing Practice (cGMP) conditions in a cost-effective and timely manner.

The compacted rods, matrices, and plates, which are substantially devoid of interconnected pores, can be ground using a cryo-milling device to produce microgranules suitable for injection. This process can also be readily adapted to cGMP conditions.

Calculation of the buprenorphine dose for a long-acting formulation requires an understanding of the minimum effective concentration (Cₘᵢₙ) for clinical efficacy. Various studies on buprenorphine pharmacokinetic studies in humans have shown that the Cₘᵢₙ is 0.1 ng/mL with the aim of 0.5˜0.7 ng/mL. This target range matches the plasma concentrations achieved by Probuphine (4˜5 implants). The mean steady-state plasma buprenorphine concentration over 6 months was approximately 0.5˜1 ng/mL. Probuphine is effective in treating OUD and clinically similar to those receiving sublingual buprenorphine/naloxone. Probuphine is indicated for the maintenance treatment of opioid dependence in patients who have achieved and sustained prolonged clinical stability on low-to-moderate doses of a transmucosal buprenorphine product. The effective buprenorphine concentrations in various animals, including common marmosets, dogs, mice, and rats, are similar. FDA has released “Opioid Use Disorder: Developing Buprenorphine Depot Products for Treatment,” a guidance document encouraging widespread innovation and development of new buprenorphine-based treatments for OUD.

Probuphine delivers 296.8 mg of buprenorphine for 6 months. Therefore, for formulations delivering buprenorphine for 3 months, for example, 150 mg buprenorphine may be sufficient to maintain the Cₘᵢₙ for 3 months. However, the 3-month dose can be significantly reduced if the initial burst release occurring hours after administration for a few days can be prevented or reduced. Surprisingly, such a dose can be used to extend the duration longer than 3 months. Furthermore, when the Norvex microparticle formulation was tested in humans, 1.5 mg/day was established as a reasonable starting dose by the FDA. Thus, 150 mg for a 3-month formulation provides a realistic initial target dose.

The advantages of compacted single-rod and microgranule formulations include high buprenorphine loading, controllable drug release kinetics, and the simpler processing steps (e.g., no dissolution of PLGA in solvents and no solvent extraction in aqueous media) compared to emulsion-based microparticle systems. Compacted single-rod and microgranule formulations, as described herein, provide an additional, longer-acting treatment option for physicians and patients alike. Such increased access to buprenorphine medication for ≥3 months by a single administration provides one of the most effective and important ways to treat OUDs.

For treatment purposes, pharmaceutical compositions comprising the compounds described herein may further comprise one or more pharmaceutically acceptable excipients. A pharmaceutically acceptable excipient is a substance that is non-toxic and otherwise biologically suitable for administration to a subject. Such excipients facilitate the administration of the compounds described herein and are compatible with the active ingredient. Examples of pharmaceutically acceptable excipients include lubricants, stabilizers, surfactants, buffers, diluents, antioxidants, binders, and coloring agents. In preferred embodiments, pharmaceutical compositions according to the invention are sterile compositions. Pharmaceutical compositions may be prepared using compounding techniques known or that become available to those skilled in the art.

In certain embodiments, the drug is preferably a drug that is advantageously delivered for a prolonged period of time or may be one that has poor compliance. In certain embodiments, the drug is a small molecule, such as an opioid-antagonist (e.g., buprenorphine or a salt thereof, naltrexone or a salt thereof, or nalmefene or a salt thereof), a peptide (e.g., a GLP-1 inhibitor, exenatide, or an anti-cancer drug such as leuprolide), a protein (e.g., insulin or antibody), or nucleic acid (e.g., DNA or RNA). In certain embodiments, the dosage of drug can be adjusted by changing the size of solid formulation (e.g., decreasing the length or diameter of a compacted rod) or adjusting the number of microgranules.

In certain embodiments, the formulation comprises about 40% to about 80% by weight of the drug. For example, in some embodiments, the formulation comprises about 50% to about 70% by weight of the drug.

In certain embodiments, the formulation comprises about 20% to about 60% by weight of the biodegradable polymer. For example, the formulation may comprise about 30% to 50% by weight of the biodegradable polymer.

In certain embodiments, the biodegradable polymer is poly(lactide-co-glycolide) (PLGA). The ratio of lactide to glycolide in the polymer can be described as a L:G ratio. In some embodiments, the PLGA has a lactide:glycolide (L:G) ratio of about 100:0 to about 0:100. A PLGA with an L:G ratio of 100:0 indicates polylactide and an L;G ratio of 0:100 indicates polyglycolide. In some embodiments, the PLGA has an (L:G) ratio of about 95:5 to about 5:95. In some embodiments, the PLGA has an L:G ratio of about 90:10 to about 50:50 (e.g., about 50:50, about 75:25, or about 85:15). In certain embodiments, adjusting the L:G ratio has an effect on the duration of release of the drug and the drug release kinetics.

In some embodiments, the PLGA includes an end-cap. For example, the PLGA can include an acid end-cap or an ester end-cap. In some embodiments, the PLGA includes an acid end-cap. In some embodiments, the PLGA includes an ester end-cap. In some embodiments, the end-cap may affect the degradation rate of the composition. In some embodiments, the end-cap may affect the composition, for example an ester end-cap is overall more hydrophobic than the acid end-cap when all other conditions are equal. Thus, in some embodiments, the end-cap may contribute to degradation rate and/or water absorption.

In some embodiments, the formulation provides a sustained release of the drug. For example, in some embodiments, the formulation can provide sustained release 30 days or longer, 60 days or longer, or 90 days or longer. In some embodiments, the formulation provides a sustained release of the drug for about 30 days to about 180 days, about 60 days to about 180 days, or about 90 days to about 180 days. In some embodiments, the formulation provides a sustained release of the drug for about 30 days to about 120 days, about 60 days to about 120 days, or about 90 days to about 120 days. In some embodiments, the formulation provides a sustained release of the drug for about 30 days to about 90 days or about 60 days to about 90 days.

In certain embodiments the formulation is a single rod, for example a compacted single rod. In some embodiments, the formulation is in the form of multiple rods, for example compacted multiple rods. In certain embodiments, the rods (e.g., single or multiple) are capable of being implanted or injected in an animal (e.g., a human) for delivery of the drug.

In certain embodiments, the formulation is in the form of a granule (e.g., a microgranule such as a compacted microgranule). In certain embodiments, the granules are capable of being injected in an animal (e.g., a human) for delivery of the drug.

In certain embodiments, the formulations described herein contain a lubricant. The lubricant can be present at amounts of about 0.01% to about 1% w/w or about 0.1% to about 1%, for example about 0.5% w/w. In certain embodiments, the lubricant is magnesium stearate.

In certain embodiments, a method of treating a patient in need thereof comprises administering to the patient a biodegradable formulation as described herein. In some embodiments, the formulation is injected (e.g., a compacted microgranule is injected). In some embodiments, the formulation is implanted (e.g., implanting a single or multiple compacted rods). In some embodiments the method of treatment is a method of treating an opioid disorder.

In certain embodiments, a formulation includes a drug (e.g., buprenorphine or a salt thereof) present at about 50% to about 80% w/w (e.g., about 70%) and a biodegradable polymer (e.g., PLGA having an L:G ratio of 75:25 or 85:15) present at about 20% to about 50% w/w (e.g., about 30%) and optionally a lubricant (e.g., magnesium stearate) present at about 0.5% w/w. In certain embodiments, these formulations are capable of providing sustained release of the drug for over 90 days (e.g., for at least about 105 days or 112 days). In certain embodiments, these formulations are capable of providing sustained release of the drug for over 90 days (e.g., for at least about 120 days, or about 90 days to about 180 days).

In some embodiments, a method of making a biodegradable formulation according to the present disclosure comprises:
providing a mixture of a drug and one or more biodegradable polymers;
compacting the mixture at a first pressure and at a first temperature thereby forming a compacted mixture; and
extruding the compacted mixture at a second pressure to form a biodegradable formulation that is substantially free of interconnected pores.

In some embodiments, the first pressure is about 40 psi to about 400 psi.

In some embodiments, the second pressure is about 20 psi to about 300 psi. For example, the second pressure can be about 40 psi, about 80 psi, or about 120 psi.

In some embodiments, the first temperature is between the glass transition temperature of the biodegradable polymer and the melting temperature of each of the biodegradable polymer and the drug. For example, in some embodiments, the first temperature is about 140° C. to about 200° C. (e.g., about 140° C., about 150° C., about 160° C., about 170° C., about 175° C., about 180° C., about 185° C., about 190° C., about 195° C., or about 200° C.).

In some embodiments, the step of compacting is performed for a time to substantially remove interconnected pores from the mixture. For example, in some embodiments, the step of compacting is performed for at least one minute (e.g., about 1 minute to about 15 minutes, about 2 minutes to about 10 minutes, or about 2 minutes to about 5 minutes).

In some embodiments, the method further comprises cooling the formulation after the extruding step, thereby forming a solid biodegradable formulation that is substantially free of interconnected pores (e.g., free of internal and external pores). In certain embodiments, the step of cooling yields the compacted rods as described herein.

In some embodiments, the method further comprises cryo-milling the biodegradable compacted formulation to produce compacted microgranules. In certain embodiments, the microgranules are passed through a sieve, for example, a 150 μm sieve.

In some embodiments, a method of making a biodegradable compacted formulation comprises providing a mixture of a drug and one or more biodegradable polymers;
compacting the mixture with sufficient mechanical force and applying heat above the glass transition temperature of the polymer to below the melting temperatures of the polymer and drug to remove surface pores and a substantial amount of interconnected pores; and
applying heat to sustainably remove remaining internal interconnected pores, thereby producing a biodegradable formulation that is substantially free of interconnected pores.

Additional embodiments, features, and advantages of the disclosure will be apparent from the detailed description and through practice of the disclosure. The compositions and formulations of the present disclosure can be described as embodiments in any of the following enumerated embodiments. It will be understood that any of the embodiments described herein can be used in connection with any other embodiments described herein to the extent that the embodiments do not contradict one another.
1. A biodegradable formulation comprising a drug and one or more biodegradable polymers, wherein the formulation is essentially free of interconnected pores.
2. The biodegradable formulation of embodiment 1, that comprises about 40 to about 80% by weight of the drug.
3. The biodegradable formulation of embodiment 1, that comprises about 50 to about 70% by weight of the drug.
4. The biodegradable formulation of any one of embodiments 1-3, wherein the drug is buprenorphine.
5. The biodegradable formulation of embodiment 4, wherein the buprenorphine is of the free base form, salt form, or mixtures thereof.
6. The biodegradable formulation of any one of the preceding embodiments, wherein the formulation comprises about 20 to about 60% by weight of the biodegradable polymer.
7. The biodegradable formulation of any one of the preceding embodiments, wherein the formulation comprises about 30 to 50% by weight of the biodegradable polymer.
8. The biodegradable formulation of any one of the preceding embodiments, wherein the biodegradable polymer is poly(lactide-co-glycolide), poly(D,L-lactide), poly(ε-caprolactone), polyhydroxybutyrate, polyanhydrides, polyorthoesters, or combinations of any of these.
9. The biodegradable formulation of embodiment 8, wherein the poly(lactide-co-glycolide) has a lactide:glycolide (L:G) ratio of about 95:5 to about 5:95 (e.g., about 50:50, about 75:25, or about 85:15).
10. The biodegradable formulation of embodiment 8, wherein the poly(lactide-co-glycolide) has a lactide:glycolide (L:G) ratio of about 90:10 to about 50:50 (e.g., about 50:50, about 75:25, or about 85:15).
11. The biodegradable formulation of any one of the preceding embodiments, wherein the formulation provides sustained release of the drug for about 90 days or longer.
12. The biodegradable formulation of any one of the preceding embodiments, wherein the surface of the biodegradable formulation is free of interconnected pores.
13. The biodegradable formulation of any one of the preceding embodiments, wherein the formulation is in the form of a compacted single rod.
14. The biodegradable formulation of any one of embodiments 1 to 12, wherein the formulation is in the form of compacted multiple rods.
15. The biodegradable formulation of any one of the preceding embodiments, wherein the formulation is implantable.
16. The biodegradable formulation of any one of embodiments 1 to 12, wherein the formulation is in the form of compacted microgranules.
17. The biodegradable formulation of any one of embodiments 1-12 or 16, wherein the formulation is injectable.
18. A method of treating a subject with an opioid use disorder, the method comprising: administering to a subject in need thereof, a biodegradable formulation according to any one of embodiments 1-17.
19. The method of embodiment 18, wherein the formulation is implanted into the patient.
20. The method of embodiment 18, wherein the formulation is injected into the patient.
21. A method of making a biodegradable formulation according to any one of embodiments 1-17, the method comprising:
   providing a mixture of a drug and one or more biodegradable polymers;
   compacting the mixture at a first pressure and at a first temperature thereby forming a compacted mixture;
   extruding the compacted mixture at a second pressure to form a biodegradable formulation that is substantially free of interconnected pores,
   wherein the first temperature is between the glass transition temperature of the biodegradable polymer and the melting temperature of each of the biodegradable polymer and the drug.
22. The method of embodiment 21, wherein the first pressure is about 40 psi to about 400 psi.
23. The method of embodiment 22 or 23, wherein the second pressure is about 20 psi to about 300 psi (e.g., about 40 psi, about 80 psi, or about 120 psi).
24. The method of any one of embodiments 21-23, wherein the first temperature is about 140° C. to about 200° C. (e.g., about 140° C., about 150° C., about 160° C., about 170° C., about 175° C., about 180° C., about 185° C., about 190° C., about 195° C., or about 200° C.).
25. The method of any one of embodiments 21-24, wherein the step of compacting is performed for a time to substantially remove interconnected pores from the mixture.
26. The method of any one of embodiments 21-25, wherein the step of compacting is performed for at least one minute (e.g., about 1 minute to about 15 minutes, about 2 minutes to about 10 minutes, or about 2 minutes to about 5 minutes).
27. The method of any one of embodiments 21-26, further comprising cooling the formulation after the extruding step thereby forming a solid biodegradable formulation that is substantially free of interconnected pores (e.g., free of internal and external interconnected pores).
28. The method of any one of embodiments 21-27, further comprising a step of cryo-milling the biodegradable formulation to produce compacted microgranules.
29. A method of making a biodegradable formulation according to any one of embodiments 1-17, the method comprising:
   providing a mixture of a drug and one or more biodegradable polymers;
   compacting the mixture with sufficient mechanical force and applying heat above the glass transition temperature of the polymer to below the melting temperatures of the polymer and drug to remove a substantial amount of surface and internal interconnected pores; and
   applying heat to sustainably remove remaining interconnected internal pores, thereby producing a biodegradable formulation that is substantially free of interconnected pores.
30. A method of treating a patient in need thereof, the method comprising,
   administering to the patient a biodegradable formulation according to any one of embodiments 1-17 or produced by a method according to any one of embodiments 18-29.
31. The method of embodiment 30, wherein the step of administering is performed by implanting the biodegradable formulation.
32. The method of embodiment 30, wherein the step of administering is performed by injecting the biodegradable formulation.

### INCORPORATION BY REFERENCE

References and citations to other documents, such as patents, patent applications, patent publications, journals, books, papers, and web contents, have been made throughout this disclosure, including to the Supplementary. The Supplementary and all other such documents are hereby incorporated herein by reference in their entirety for all purposes.

### EQUIVALENTS

The invention may be embodied in other specific forms without departing from the spirit or essential characteristics thereof. The foregoing embodiments are, therefore, to be considered in all respects illustrative rather than limiting to the invention described herein.

### EXAMPLES

### Example 1: Compacted Buprenorphine Rod Formation

Compacted rod-shaped formulations were manufactured using a plastometer (also known as an extrusion plastometer or a melt flow index tester, Tinius Olsen MP1200) with slight modifications to the die geometry.

Traditionally, a plastometer has been used to measure the flow characteristics of thermoplastic polymers. Specifically, it determines how easily a polymer melts and flows under a given set of conditions (e.g., temperature, weight, or load), indicating its viscosity. The melt flow index value provides a value to evaluate the processability of a polymer for different applications (e.g., injection molding or extrusion). A plastometer consists of a barrel with a heating chamber where the polymer sample is placed and melted in a heated barrel. A piston is used to apply a certain amount of pressure on the melted polymer in the barrel. The pressure forces the polymer through a small die orifice placed at the end of the barrel. The force applied by the piston is constant, and the subsequent flow of the polymer is measured. The size and length of the orifice are standardized according to the respective ASTM method used, and the rate of flow is used to calculate the MFI value.

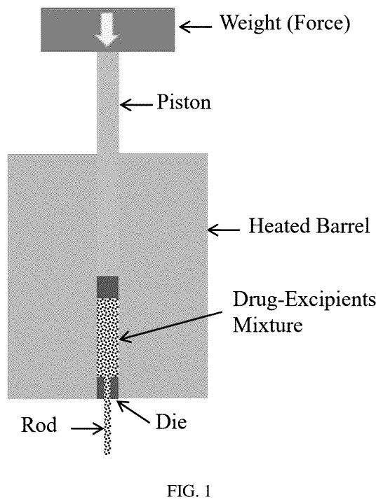

For making compacted buprenorphine rod formulations in this example, the barrel temperature used was always lower than the melting temperatures of the drug and polymer in the formulation. The pre-compaction load, compaction load, die orifice and length, barrel temperature, and barrel diameter were adjusted in the plastometer according to the respective formulation being prepared. FIG. 1 illustrates an overall approach to manufacturing buprenorphine/PLGA rod formation using a plastometer.

### Example 2: Preparation of Buprenorphine Free Base

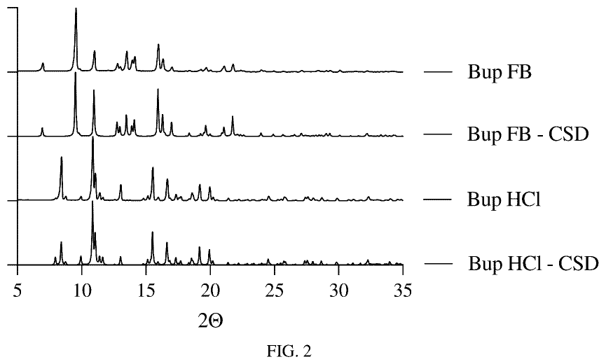

Buprenorphine free base (FB) was created by dissolving buprenorphine HCl in water with 3% v/v methanol to obtain a 1.3% solution. Buprenorphine FB was precipitated with excess NaOH. Dichloromethane at a 13% v/v ratio was then added to dissolve the precipitated buprenorphine, and subsequently, a liquid-liquid extraction was performed to remove the dichloromethane-buprenorphine solution. The dichloromethane was subsequently removed by vacuum over 48 hours, resulting in buprenorphine free base. The FB and HCl salt forms were compared to those in the Cambridge Structural Database (CSD). FIG. 2 illustrates the Powder X-ray diffraction patterns from the CSD compared to the HCl and FB forms used in the following examples.

### Example 3: Solubility of Buprenorphine Free Base

The solubility of buprenorphine FB was determined in PBS with 0.05% Tween 20 (PBST) and 0.5% w/v sodium dodecyl sulfate (SDS) in water. The solubility was determined by placing excess buprenorphine FB into the media mentioned above at 37° C. and shaking at 40 RPM for 48 hours. After 48 hours, a sample was taken and centrifuged at 15,000 RPM for 5 minutes. The supernatant was then collected and diluted with the same medium, and the concentration was determined using high-performance liquid chromatography. The solubility values are summarized in Table 1.

TABLE 1
Solubility of Buprenorphine FB in PBS, PBST, and 0.5% SDS

| | PBST | 0.5% SDS |
|---|---|---|
| Buprenorphine FB | 0.0088 mg/mL | 0.336 mg/mL |

### Example 4: Preparation of Buprenorphine Rods without PLGA

Buprenorphine rods were prepared using a plastometer. Buprenorphine FB powder was blended with magnesium stearate (0.5% w/w) on a Resodyn LabRam II acoustic mixer for 2.5 minutes at 80 g. A die with an orifice of 3.5 mm was used for the rod preparation. One gram of material was loaded into the plastometer set at a predetermined temperature (listed in Table 2) with the die plugged. The piston was then inserted into the heated barrel, and a precompaction weight of 16 kg was applied for 3 minutes. This precompaction weight eliminated empty spaces from the powder mixture, creating a solid entity. The precompaction weight was removed, followed by the removal of the die plug. A 10 kg weight was then applied to generate a buprenorphine rod. As listed in Table 2, buprenorphine alone without PLGA could not form rods, and as the temperature increased to 210° C., melted buprenorphine flew through the orifice.

TABLE 2
Lack of the rod formation of buprenorphine FB without PLGA.

| Temperature (° C.) | Result |
|---|---|
| 160 | No rod formation, no flow from the orifice |
| 170 | No rod formation, no flow from the orifice |
| 180 | No rod formation, no flow from the orifice |
| 190 | No rod formation, no flow from the orifice |
| 200 | No rod formation, no flow from the orifice |
| 210 | No rod formation, flow of melted buprenorphine |

### Example 5: Preparation of PLGA Powder

PLGA is typically supplied as large granules, with the granules often being larger than the orifice of 2.095 mm used for ASTM test D1238 of the plastometer. To facilitate thorough mixing with buprenorphine, smaller PLGA particles were prepared by cryo-milling.

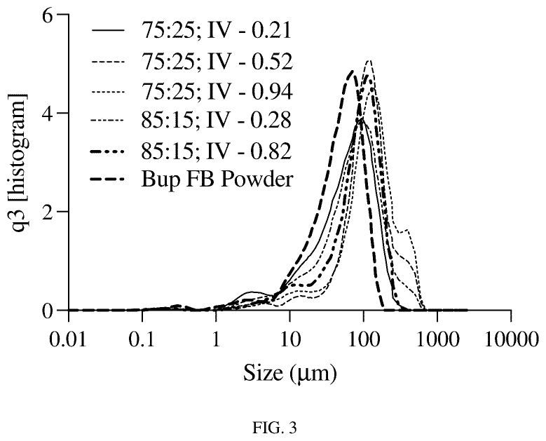

A Retsch CryoMill was used to prepare PLGA particles of various grades of ester end-capped PLGA (Ashland), as summarized in Table 3. About 10 mL of tapped PLGA granules was measured and placed into a 25 mL stainless steel CryoMill jar with 10 stainless steel media balls (8 mm diameter). The jar was precooled for 10 minutes, followed by 6 cycles of 10 minutes of milling at a frequency of 30 Hz, with an immediate 1-minute intermediate cooling time between each cycle. The ground PLGA was allowed to equilibrate to room temperature, followed by passing it through a 150 μm sieve and collecting it for use. The particle size distributions of representative PLGA samples are shown in FIG. 3.

Fine buprenorphine powder was prepared by grinding buprenorphine FB by hand using a mortar and pestle. The ground material was then passed through a 150 μm sieve and collected for use.

TABLE 3
Particles of ester end-capped PLGA from Ashland.

| | Inherent Viscosity |
|---|---|
| L:G ratio | (dL/g) |
| 75:25 | 0.21 |
| 75:25 | 0.52 |
| 75:25 | 0.94 |
| 85:15 | 0.28 |
| 85:15 | 0.82 |

### Example 6: Impact of L:G Ratio on Pharmacokinetics and Degradation in Rodents

Compacted buprenorphine rods were prepared using a plastometer. Buprenorphine FB powder (70% w/w) was blended with PLGA powder (30% w/w) with a Resodyn LabRam II acoustic mixer for 1 minute at 80 g. Magnesium stearate (0.5% w/w) was added and then blended on the Resodyn mixer with the same settings for 2.5 minutes. A die with an orifice of 3.5 mm was used for the rod preparation. One gram of material was loaded into the plastometer set at a predetermined temperature (listed in Table 4) with the die plugged. The temperatures used for precompaction were all below the melting temperatures of the polymer and drug. The piston was then inserted into the heated barrel, and a precompaction weight of 16 kg was applied for 3 minutes. This precompaction weight eliminated empty spaces from the powder mixture, creating a solid entity. The precompaction weight was removed, followed by the removal of the die plug. Then, a weight of 4, 6, or 10 kg was applied to generate a buprenorphine-loaded rod. The rods were allowed to cool to room temperature and then cut to size. The temperature and weight were chosen to provide a similar rod flow rate across formulations.

TABLE 4
Three buprenorphine rod formulations used in the rodent study.

| | | Inherent Viscosity | Temperature | |
|---|---|---|---|---|
| Formulation ID | L:G ratio | (dL/g) | (° C.) | Weight (kg) |
| F1 | 75:25 | 0.21 | 180 | 4 |
| F2 | 85:15 | 0.28 | 165 | 6 |
| F3 | 85:15 | 0.82 | 180 | 10 |

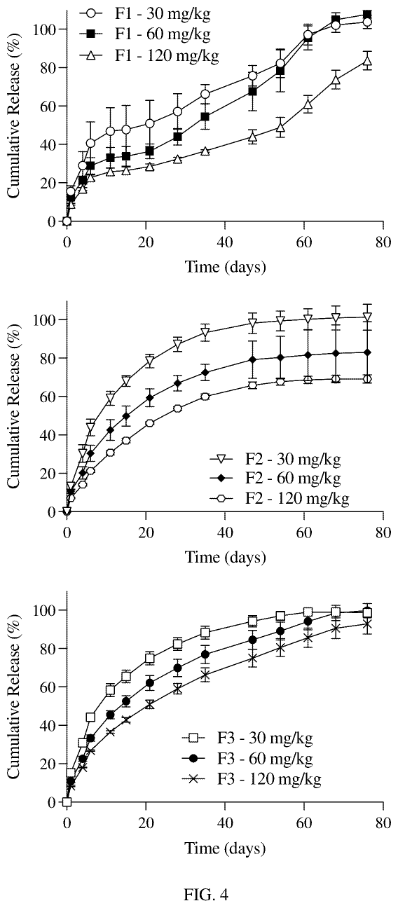

Compacted buprenorphine rods (30, 60, or 120 mg/kg buprenorphine) corresponding to a rodent weight of 300 g were characterized by an in vitro release method. 50 mL of 0.5% sodium dodecyl sulfate was placed into 125 mL Erlenmeyer flasks with rubber stoppers, and compacted rods were individually placed into flasks (n=3). Release samples were taken frequently, and the entire release medium was replaced with fresh media. Representative buprenorphine concentrations in the release media were determined by HPLC. The in vitro release profiles of the formulations described in Table 4 are shown in FIG. 4.

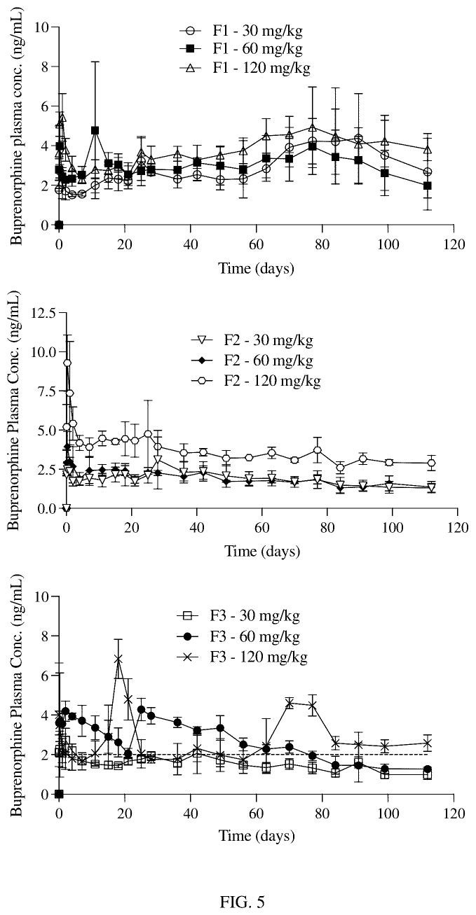

For in vivo studies, compacted buprenorphine rods (30, 60, or 120 mg/kg buprenorphine, n=3) were placed in the subcutaneous space in the dorsal scruff region by a 3.5 mm trocar and cannula under sterile conditions. Blood samples were collected from the cephalic vein at various time points over a period of 112 days. Plasma concentrations of buprenorphine were quantified using liquid chromatography-tandem mass spectrometry (LC/MS/MS). The pharmacokinetic profiles of the formulations described in Table 4 are shown in FIG. 5. After 112 days, any residual buprenorphine rods were removed and quantified for the remaining buprenorphine by HPLC. The remaining buprenorphine from the formulations administered to the rodents after 112 days is listed in Table 5. As shown in the table, substantial amounts of the loaded buprenorphine remained in the buprenorphine rods recovered after 112 days. Thus, it indicates that the buprenorphine release can be extended much longer than 112 days.

TABLE 5
The residual buprenorphine (%) remained in the recovered rods.

| Dose (mg/kg) | F1 | F2 | F3 |
|---|---|---|---|
| 30 | 46.3 ± 11.8 | 78.5 ± 9.7 | 67.2* |
| 60 | 72.0 ± 5.1 | 79.0 ± 1.9 | 84.7 ± 3.4 |
| 120 | 81.8 ± 1.7 | 91.6 ± 1.2 | 90.0 ± 2.5 |

*n = 2, Value listed is average ± standard deviation.

### Example 7: Dog Pharmacokinetic Study I

Compacted buprenorphine rods were prepared using a plastometer. Buprenorphine free base powder (70% w/w) was blended with PLGA powder (30% w/w) with a Resodyn acoustic mixer for 1 minute. Magnesium stearate (0.5% w/w) was added and then blended on the Resodyn mixer with the same settings for 5 minutes. A die with an orifice of 3.5 mm was used for the rod preparation. One gram of material was loaded into the plastometer, set at a predetermined temperature (listed in Table 6), with the die plugged. The temperatures used for precompaction were all below the melting temperatures of the polymer and drug. The piston was then inserted into the heated barrel, and a precompaction weight of 16 kg was applied for 3 minutes. This precompaction weight eliminated empty spaces from the powder mixture to create a solid entity. The precompaction weight was removed, followed by the removal of the die plug, and then a weight was applied to generate a buprenorphine-loaded rod. The temperature and weight were chosen to provide a similar rod flow rate across formulations. The rods were allowed to cool to room temperature and then cut to size.

TABLE 6
Five buprenorphine rod formulations used in the dog study.

| | | Inherent | | |
|---|---|---|---|---|
| Formulation | | Viscosity | Temperature | Weight |
| ID | L:G ratio | (dL/g) | (° C.) | (kg) |
| F1 | 75:25 | 0.21 | 180 | 4 |
| F2 | 75:25 | 0.52 | 180 | 10 |
| F3 | 75:25 | 0.94 | 180 | 10 |
| F4 | 85:15 | 0.28 | 180 | 6 |
| F5 | 85:15 | 0.82 | 165 | 10 |

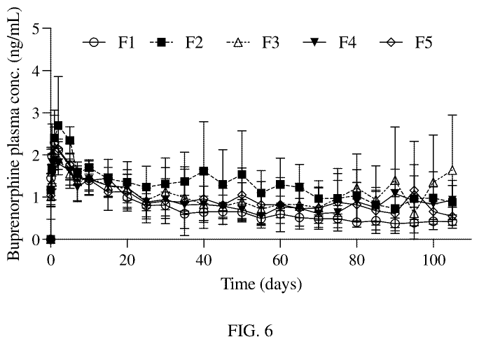

Compacted buprenorphine rods (250 mg buprenorphine, n=4) were placed in the subcutaneous space in the cranial region of beagle dogs via a trocar and cannula under sterile conditions. Blood samples were collected from the cephalic vein at various time points over a period of 105 days. Plasma concentrations of buprenorphine were quantified using LC/MS/MS. The pharmacokinetic profiles of the three formulations described in Table 6 are shown in FIG. 6. After 105 days, any residual buprenorphine rods were removed and quantified for the remaining buprenorphine by HPLC. The remaining buprenorphine from the formulations administered to the dogs after 105 days is listed in Table 7.

TABLE 7
Residual buprenorphine (%) remaining in the recovered rods.

| | Remaining Buprenorphine |
|---|---|
| Formulation ID | (%) |
| F1 | 80.8 ± 2.7 |
| F2 | 61.0 ± 14.3 |
| F3 | 68.3 ± 14.9 |
| F4 | 68.7 ± 15.0 |
| F5 | 75.1 ± 6.0 |

### Example 8: In Vitro Release Comparison in Different Media

Compacted buprenorphine rods were prepared using a plastometer. Buprenorphine FB powder (70% w/w) was blended with PLGA powder (30% w/w) with a Resodyn acoustic mixer for 1 minute. Magnesium stearate (0.5% w/w) was added and then blended on the Resodyn mixer with the same settings for 5 minutes. A die with an orifice of 3.5 mm was used for the rod preparation. One gram of material was loaded into the plastometer, set at a predetermined temperature (listed in Table 8), with the die plugged. The temperatures used for precompaction were all below the melting temperatures of the polymer and drug. The piston was then inserted into the heated barrel, and a precompaction weight of 16 kg was applied for 3 minutes. This precompaction weight eliminated empty spaces from the powder mixture to create a solid entity. The precompaction weight was removed, followed by the removal of the die plug, and then a weight was applied to generate a buprenorphine-loaded rod. The temperature and weight were chosen to provide a similar rod flow rate across formulations. The rods were allowed to cool to room temperature and then cut to size.

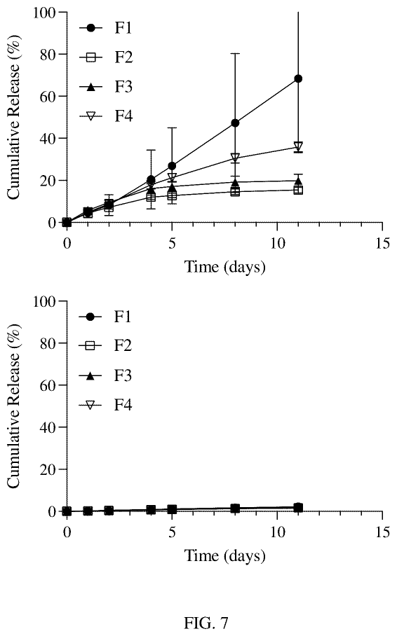

Compacted buprenorphine rods (˜70 mg) were characterized using in vitro release. 50 mL of 0.5% sodium dodecyl sulfate or phosphate-buffered saline with 0.05% Tween® 20 was placed into 125 mL Erlenmeyer flasks with rubber stoppers, and compacted rods were individually placed into flasks (n=3). Release samples were taken frequently, and the entire release medium was replaced with fresh media. Representative buprenorphine concentrations in the release media were determined by HPLC. The in vitro release profiles of the formulations described in Table 8 are shown in FIG. 7. Buprenorphine was released from the rods in 0.5% SDS (Top of FIG. 7) but not in 0.05% PBST (Bottom of FIG. 7).

TABLE 8
Four buprenorphine rod formulations used in the study.

| Formu- | | PLGA | Inherent | Tem- | |
|---|---|---|---|---|---|
| lation | L:G | End- | Viscosity | perature | Weight |
| ID | ratio | Cap | (dL/g) | (° C.) | (kg) |
| F1 | 50:50 | Acid | 0.15 | 180° C. | 10 kg |
| F2 | 50:50 | Acid | 0.46 | 180° C. | 10 kg |
| F3 | 75:25 | Ester | 0.21 | 165° C. | 4 kg |
| F4 | 85:15 | Ester | 0.82 | 165° C. | 10 kg |

### Example 9: Dog Pharmacokinetic Study II

Compacted buprenorphine rods were prepared using a plastometer. Buprenorphine free base powder (70% w/w) was blended with PLGA powder (30% w/w) with a Resodyn acoustic mixer for 1 minute. Magnesium stearate (0.5% w/w) was added and then mixed on the Resodyn mixer with the same settings for 5 minutes. A die with an orifice of 3.5 mm was used for the rod preparation. One gram of material was loaded into the plastometer, set at a predetermined temperature (listed in Table 9), with the die plugged. The temperatures used for precompaction were all below the melting temperatures of the polymer and drug. The piston was then inserted into the heated barrel, and a precompaction weight of 16 kg was applied for 3 minutes. This precompaction weight eliminated empty spaces from the powder mixture to create a solid entity. The precompaction weight was removed, followed by the removal of the die plug, and then a weight was applied to generate a buprenorphine-loaded rod. The temperature and weight were chosen to provide a similar rod flow rate across formulations. The rods were allowed to cool to room temperature and then cut to size.

TABLE 9
Five buprenorphine rod formulations used in the dog study.

| | | PLGA | | Inherent | | |
|---|---|---|---|---|---|---|
| Formulation | L:G | End- | Buprenorphine | Viscosity | Temperature | Weight |
| ID | ratio | Cap | Form | (dL/g) | (° C.) | (kg) |
| F1 | 50:50 | Acid | Free Base | 0.15 | 180 | 10 |
| F2 | 50:50 | Acid | HCl Salt | 0.15 | 180 | 10 |
| F3 | 50:50 | Ester | HCl Salt | 0.17 | 180 | 10 |
| F4 | 65:35 | Ester | HCl Salt | 0.21 | 180 | 6 |
| F5 | 75:25 | Ester | HCl Salt | 0.21 | 165 | 4 |

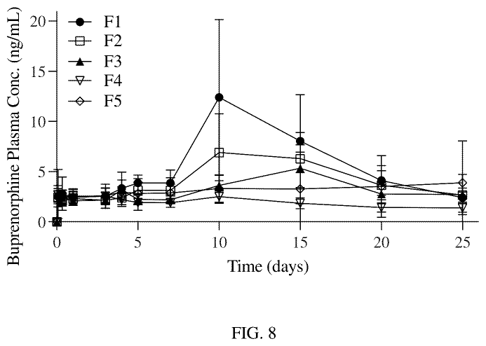

Compacted buprenorphine rods (300 mg buprenorphine, n=4) were placed in the subcutaneous space in the cranial region of beagle dogs via a trocar and cannula under sterile conditions. Blood samples were collected from the cephalic vein at various time points. Plasma concentrations of buprenorphine were quantified by LC/MS/MS. The pharmacokinetic profiles of the formulations described in Table 9 are shown in FIG. 8. The duration of release may be shortened and/or steady-state plasma concentration may be increased with a lower L:G ratio PLGA (e.g., 50:50) and/or the use of the HCl form of buprenorphine. Patients in treatment for OUD may require different steady-state plasma concentrations of buprenorphine for treatment. Those in treatment for moderate to severe OUD may require higher steady-state buprenorphine levels than those who have achieved clinical stability on low-to-moderate doses of oral buprenorphine.

### Example 10: Preparation of Compacted Buprenorphine Microgranules

Compacted buprenorphine microgranules were prepared by grinding compacted buprenorphine rods prepared for rodent and dog studies by cryo-milling using Retsch CryoMill. The rods were broken into smaller pieces before placing into a 25 mL stainless steel CryoMill jar with 10 stainless steel media balls (8 mm diameter). The jar was precooled for 10 minutes, followed by 6 cycles of 10 minutes of milling at a frequency of 30 Hz, with an immediate 1-minute intermediate cooling time between each cycle. The obtained microgranules were allowed to equilibrate to room temperature, then passed through a 150 μm sieve and collected for use.

What is claimed is:

1. A compacted biodegradable formulation comprising:
a drug and
one or more biodegradable polymers,
wherein the formulation is essentially free of interconnected pores, and
wherein the drug is a small molecule, a peptide, a protein, or a nucleic acid.

2. The biodegradable formulation of claim 1, comprising about 40% to about 80% by weight of the drug.

3. The biodegradable formulation of claim 2, wherein the drug is a small molecule.

4. The biodegradable formulation of claim 3, wherein the drug is buprenorphine, a free base form thereof, a salt form thereof, or mixtures thereof.

5. The biodegradable formulation of claim 2, wherein the formulation comprises about 20% to about 60% by weight of the biodegradable polymer.

6. The biodegradable formulation of claim 5, wherein the biodegradable polymer is poly(lactide-co-glycolide), poly(D,L-lactide), poly(ε-caprolactone), polyhydroxybutyrate, polyanhydrides, polyorthoesters, or combinations of any of these.

7. The biodegradable formulation of claim 6, wherein the poly(lactide-co-glycolide) has a lactide:glycolide (L:G) ratio of about 95:5 to about 5:95.

8. The biodegradable formulation of claim 1, wherein the formulation provides sustained release of the drug for about 90 days or longer.

9. The biodegradable formulation of claim 8, wherein the formulation is in the form of a compacted single rod.

10. The biodegradable formulation of claim 1, wherein the formulation is in the form of compacted microgranules.

11. The biodegradable formulation of claim 10, wherein the formulation is injectable.

12. A method of treating a subject with an opioid use disorder, the method comprising: administering a biodegradable formulation according to claim 1 to the subject in need thereof.

13. A method of making a compacted biodegradable formulation according claim 1, the method comprising:
providing a mixture of a drug and one or more biodegradable polymers;
compacting the mixture at a first pressure and at a first temperature thereby forming a compacted mixture;
extruding the compacted mixture at a second pressure to form a biodegradable formulation that is substantially free of interconnected pores,
wherein the first temperature is between the glass transition temperature of the biodegradable polymer and the melting temperature of each of the biodegradable polymer and the drug.

14. The method of claim 13, wherein the first pressure is about 40 psi to about 400 psi.

15. The method of claim 13, wherein the second pressure is about 20 psi to about 300 psi.

16. The method of claim 13, wherein the first temperature is about 140° C. to about 200° C.

17. The method of claim 13, wherein the step of compacting is performed for a time to substantially remove interconnected pores from the mixture.

18. The method of claim 13, wherein the step of compacting is performed for at least one minute.

19. The method of claim 13, further comprising cooling the formulation after the extruding step thereby forming a solid biodegradable formulation that is substantially free of interconnected pores.

20. A method of making a biodegradable formulation according to claim 1, the method comprising:
providing a mixture of a drug and one or more biodegradable polymers;
compacting the mixture with sufficient mechanical force and applying heat above the glass transition temperature of the polymer to below the melting temperatures of the polymer and drug to remove a substantial amount of surface and internal interconnected pores; and
applying heat to sustainably remove remaining interconnected internal pores, thereby producing a biodegradable formulation that is substantially free of interconnected pores.
